(19) United States
(12) Patent Application Publication
(10) Pub. No.: US 2023/0301222 A1
(43) Pub. Date: Sep. 28, 2023

(54) Method for Monitoring the Mechanical, Automated Removal of Weeds

(71) Applicant: Amazonen-Werke H. Dreyer SE & Co. KG, Hasbergen (DE)

(72) Inventor: Karl WILD, Ullersdorf (DE)

(73) Assignee: Amazonen-Werke H. Dreyer SE & Co. KG, Hasbergen (DE)

(21) Appl. No.: 18/022,899

(22) PCT Filed: Aug. 31, 2021
(86) PCT No.: PCT/EP2021/073935

§ 371 Date: Feb. 23, 2023

(30) Foreign Application Priority Data
Aug. 31, 2020 (DE) 102020210951.6

(51) Int. Cl. A01B 39/18; A01B 71/08; A01B 71/02; A01M 21/02; G01S 15/08; G01N 21/47; G01N 33/24; G06T 7/00

(52) U.S. Cl. CPC A01B 39/18; A01B 71/08; A01B 71/02; A01M 21/02; G01S 15/08; G01N 21/47; G01N 33/24; G06T 7/0002; G01N 2033/245; G06T 2207/30188

(57) ABSTRACT

A method for monitoring the mechanical automated removal of weeds comprising emitting ultrasonic waves in a direction perpendicular to the direction of the soil by means of at least one ultrasonic distance sensor, determining the distance to the soil, emitting electromagnetic radiation in the direction of the soil by means of at least one radiation source, registering the intensity of electromagnetic radiation reflected or scattered there by at least one optical detector arranged on the hoeing machine, using the registered intensity to determine whether the tillage has been sufficient to lead to a removal of weeds and/or whether too much dust has been whirled up, detecting shear and/or bending stresses at individual tools using strain gauges and comparing these stresses with calibration values, detecting airborne sound waves using at least one microphone, and comparing the registered airborne sound spectra with airborne sound spectra registered in advance in the case of a faultless weed removal.

The present application claims priority under 35 U.S.C. §365 to International Application No. PCT/EP2021/073935 filed on Aug. 31, 2021, and under 35 U.S.C. §119 to German Application Number DE 102020210951.6 filed on Aug. 31, 2020.

### BACKGROUND

The present disclosure relates to a method for monitoring the mechanical automated removal of weeds during field cultivation of crops growing in a field using a mobile hoeing machine.

Currently, in most cases, field work is performed by a combination of tractor equipment. Here, the driver controls the tractor and supervises the work process, which is done by the attached or mounted implement. In the meantime, there have also emerged initial solutions in which work can be performed using autonomous robotic vehicles. In this case, there is no need for a vehicle driver. In future, these methods will gain considerably in importance. The most common application of this new technology involves currently mechanized weed control using hoeing machines. Under optimal conditions, there can be achieved good work results. These optimal conditions however are not often available, therefore disturbances during the hoeing process may occur. These include, for example, clogging of the hoeing tools, weeds getting stuck on the hoeing tools or stones getting jammed. These disturbances not only negatively affect the performance of the hoeing operation, but can also cause considerable damage to the crop. With the common tractor-hoeing machine combination, the operator notices these disturbances and can eliminate them. In case of hoeing robots, there are currently no suitable systems for disturbance detection. Some hoeing robots are indeed equipped with a camera that can send images to the operator's smart phone. However, the operator cannot constantly monitor the hoeing process via a display screen, as this link would mean that the advantage of the driverless hoeing system would be lost to a large extent.

### SUMMARY

For this reason, there is a demand for technology that can register malfunctions and then send the operator the appropriate information. Then the user can move in and eliminate the malfunction. It would be even more advantageous if the hoeing machine could eliminate the malfunction on its own, without any operator's intervention.

Through the movement of the tools in the ground, the tools are set into vibration. The amplitude and frequency of these vibrations depend on a number of parameters, such as specific soil resistance, working depth, travel speed and, of course, the tool itself. If there comes to clogging caused by soil or weeds that are pushed up in front of the tool, the vibration behavior changes with regard to amplitude and frequency. The same also applies if a stone gets stuck or if weeds wrap around the tool (in case of rotary tools) or get stuck on the tool or if the tool/coulter gets bent or lost. If an accelerometer, ideally a 3-axis one, is now attached to the tool, these vibrations can be measured and passed on to a central unit for registration and processing. Appropriate signal analysis techniques (FFT, digital filters, etc.) can be used to register changes in vibration behavior and thus identify problems. At the beginning of the hoeing work, a calibration can first be carried out on the first approx. 100 m, i.e. the measured values are registered during disturbance-free operation. The measured values are filtered or smoothed and threshold values (lower and upper limits for frequencies and amplitudes) are created for the different vibration levels. This creates a signature for each hoeing unit. If the threshold values are exceeded or undershot for a longer period of time during hoeing, it can be assumed that a malfunction has occurred. The machine stops automatically and the farmer can be informed by message transmission, e.g. by SMS.

However, when registering and evaluating the vibrations on a hoeing machine, problems can arise in that vibrations can overlap and/or another type of vibration excitation can occur that has nothing to do with the actual hoeing operation. These can be, for example, vibrations generated as a result of accelerations occurring at the hoeing machine during the run, or the bumping of a tool against a stone, or a change in the soil type. Fault monitoring carried out in this way is still subject to too many faults.

It is therefore the object of at least one embodiment of the invention to provide means for improved and reliable detection of disturbances that may occur during mechanical autonomous removal of weeds during field cultivation of crops.

According to at least one embodiment of the invention, this problem is solved with a method

### DETAILED DESCRIPTION

In the method according to at least one embodiment of the invention, at least one of the following steps is carried out.

In step i) emitting ultrasonic waves perpendicularly in the direction of the soil with at least one ultrasonic distance sensor arranged in the region of a tool on the hoeing machine, and determining the distance to the soil surface by determining the propagation time of the emitted ultrasonic waves reflected by the soil until their detection and/or in a step ii) emitting electromagnetic radiation with at least one radiation source, which can be a light source or also the sun, in the direction of the soil into a surface region, in which mechanical weed removal is carried out or is to be carried out, and registering the intensity of electromagnetic radiation reflected or scattered there with at least one optical detector arranged on the hoeing machine, and determining with the registered intensity whether a sufficient tillage has taken place, which has resulted in weed removal, and/or whether too much dust has been whirled up, and/or

in step iii) detecting and taking into account during monitoring of shear and/or bending stresses at individual tools with strain gauges and comparing them with calibration values, which have been determined at the corresponding tool in case of a correct removal of weeds, and/or

iv) detecting airborne sound waves emitted by at least one tool as a result of weed removal with at least one microphone and comparing the registered airborne sound spectra with an electronic evaluation unit with airborne sound spectra registered in advance during faultless weed removal.

The airborne sound waves can be registered or evaluated with frequency resolution. It is also possible to use certain typical frequencies or frequency ranges for the evaluation by filtering.

The emitted airborne sound waves or vibrations also depend on the respective soil type, soil moisture, humus content, travel speed, working depth or the respective tools. Since the hoeing work and the result also depend on these parameters, conclusions can be drawn about the hoeing machine or the hoeing work via an airborne sound wave or vibration analysis.

In addition, detectors can be used to register vibrations on the individual tools with regard to their amplitude and frequency resolution and/or to detect speeds of rotating elements on tools and take them into account for function monitoring. For the detection of vibrations, there can be used known ultrasonic wave transducers or acceleration sensors. Using this vibration analysis, which was known in principle, it is possible to improve the monitoring accuracy in connection with the simultaneous implementation of at least one of the process steps i), ii) and/or iii).

In dry conditions, varying degrees of dust release may occur and too much undesirable dust on crops may whirl up. An inexpensive optical detector can be used to determine the amount of dust generated, and if too much dust has been whirled up, an appropriate control system can be used to reduce the travel speed. In principle, the whirling up of dust can be determined according to the method step ii). It is also possible to consider the respective intensity(ies) only for certain wavelengths.

Advantageously, similar measured values registered at the same times on all tools should be compared with each other. This will at least allow one to detect major differences that may at least indicate a defect or malfunction in one or more of the tools on the particular hoeing machine.

A calibration run can be carried out before the start of monitoring and/or at least one calibration run can be carried out during processing, during which measured values are registered that have been registered during functional at least almost faultless processing. These measured values recorded during a calibration run can be used as a reference for measured values subsequently registered during processing. Calibration measurement values registered during processing can be used, for example, to compensate for or take into account changing external boundary conditions, such as increased soil compaction and/or moisture.

In addition, camera images can be transmitted from the hoeing machine so that the farmer can see what type of disturbance is present. The farmer can then react accordingly, e.g. by arriving and eliminating the malfunction or starting a malfunction elimination measure, which can be carried out automatically by the hoeing machine.

Certain disturbances can be read out from the processed measurement data. Accordingly, in certain cases, the machine can then independently start a troubleshooting procedure, such as lifting the hoeing machine in case of clogging and lowering it again after a short travel (approx. 1 m). Also, preferably in the raised condition, vibrations can be coupled by a technical device into a tool or its environment at the hoeing machine with an amplitude at which a freeing of a clogging or a jamming of weeds or objects can be achieved by the respective tool. For example, a shifting frame or an unbalance device can be used to shake the hoeing machine so that clinging weeds etc. can be shaken off. A rotating mass with an unbalance can be used as a device for coupling vibrations.

Measurement data can also be registered during shaking, so that data analysis can reveal how the vibration behavior of the tools changes. On the one hand, this can improve the fault analysis and, on the other hand, detect the elimination of the fault.

Vibrations and other measured values on the hoeing tools can also be registered during turning operations at the end of the field. It may be necessary to stop briefly for this purpose. This can be used to determine whether changes have been made to the tools.

Calibration runs, as explained earlier, can also be performed in between. For this purpose, it is favorable if the farmer observes this via the video transmission to ensure that this calibration run is performed without any disturbances.

On the hoeing machine there may be at least one camera. The registered signals can be transmitted to an electronic image evaluation unit. The data evaluated with the electronic image evaluation unit can be used to monitor whether weed plants have been sufficiently covered with soil after burrowing and/or whether crop plants have been damaged.

In fact, in mechanical weed control, there are essentially three different measures: mowing, weed pulling or tillage The most effective at this is weed pulling or tillage. In order to be able to determine the control success of these two measures in each case, the determination of the weed coverage can be used. This can be determined with the aid of a camera and electronic image evaluation unit. This can be done to determine the uncovered visible weeds or parts of them before and after the measure. After tillage, weed coverage should reach zero, i.e., no weeds are visible. If, on the contrary, is pulled out, then it should be as large as possible, that is, the weed lies completely and “elongated” on the surface of the soil, so that it quickly and well withers and then dies.

During hoeing operations, crops must not be damaged or only minimally damaged. Crops should look the same after using the hoeing machine as they did before hoeing. This can also be determined with the aid of cameras and an electronic image evaluation device. For this purpose, the cameras should be arranged before and after the tools on the hoeing machine. By simply comparing the crop images before and after hoeing, it is possible to see the extent to which something has changed in the crop or whether damage has been caused.

An electronic control system is used to adjust the travel speed, the working depth and/or the distances between the tools when at least one predetermined threshold value of a measurement signal registered is exceeded or not reached.

It is also possible to determine the force and/or power required for the advancement of the hoeing machine and thus to affect the working depth of the tools and/or to adapt the travel speed to soil conditions in a locally defined manner. Knowing the set working depth of the tools, conclusions can then be drawn about the respective soil type and soil density, which help to better interpret the measurement data and also provide information about the cutting-edge sharpness of tools.

In most cases, malfunctions do not occur simultaneously on several tools of a hoeing machine, but usually only on one or two. Therefore, it is advantageous that the simultaneously registered measured values, which are registered in the region of all individual tools of a hoeing machine, are constantly compared with each other, as this allows a defective tool to be quickly identified.

If the control for the hoeing tool guidance does not work properly or the tools are not set correctly and then (larger) crops are being hoed, then rhythmically occurring signals will result in the measurement data (especially with larger/more stable crops). These can be used to detect faulty guidance or adjustment of the hoeing tools.

In order not to have to lay a cable connection to each sensor for power supply and data transfer, systems can also be used which ensure the energy supply with Energy Harvesting (e.g. by moving tools) and enable wireless data transfer (WLAN; Bluetooth etc.).

Speed sensors can also be used for rotary tools. Their signals can be evaluated accordingly, whereby the comparison of the signals from the individual tools can also quickly lead to the detection of faults on a tool.

Another variant for fault detection would be the use of ultrasonic distance sensors (US sensors). Clogging or attached weeds will cause soil or plant material to accumulate. Vertically arranged US sensors can determine these accumulations and detect corresponding faults. Data acquisition and analysis (calibration, etc.) are then performed as already presented above. Also, the US sensors behind the hoeing tools can determine if straight larger weeds are also “lying flat.” There are also hoeing tools that mound, that is, cover the weeds with soil. Here, US sensors can be used to determine whether the mounding layer is sufficiently high and whether weeds are still protruding from the mounded soil. However, this technique can also be used to determine if crops have also been covered in an undesirable manner.

The hoeing tool monitoring system can be expanded to include additional systems for checking the quality of the hoeing work. Typically, soil in front of the hoeing tool is relatively smooth. After proper hoeing, the surface is relatively rough because the surface has been broken up. Using a radiation source that emits electromagnetic radiation and at least one optical detector, this roughness can be registered. The radiation source emits the electromagnetic radiation in the direction of the soil, from there the electromagnetic radiation is reflected and scattered to a greater or lesser extent, and reflected and scattered electromagnetic radiation impinges on at least one optical detector arranged on the hoeing machine. The rougher the soil surface, the more diffuse the reflected and scattered electromagnetic radiation from the ground, and the intensity of the reflected and scattered electromagnetic radiation registered by the one optical detector is correspondingly lower than for an untreated soil. If several optical detectors are arranged in the area of a tool, it can also be evaluated whether only a single optical detector or a larger number of optical detectors register electromagnetic radiation with an intensity that exceeds a predefined threshold value. Thereby, from a certain number of the multiple optical detectors, where this threshold is exceeded simultaneously, it can be determined that sufficient processing has taken place.

If the soil is too moist, undesirable clods will form, causing the roughness to increase sharply.

This detected excessive roughness then serves as a trigger to stop the hoeing operation or to reduce the working depth.

All registered values or signals acquired can be evaluated in an electronic data analysis device and compared with calibration measurement or reference values advantageously stored therein. Preset visual threshold values for respective measured values or measured signals can also be taken into account in the electronic data analysis device.

At least one embodiment of the invention is based on the use of appropriate sensor technology on the tools or tines of a hoeing machine and a corresponding electronic data analysis device and, if necessary, suitable actuator technology.

1. A method for monitoring the mechanical automated removal of weeds during field cultivation of crops growing on a field with a mobile hoeing machine, in which at least one of the following steps is carried out:
i) emitting ultrasonic waves perpendicularly in the direction of soil with at least one ultrasonic distance sensor arranged in a region of a tool on the hoeing machine, and determining the distance to the soil surface by determining the propagation time of the emitted ultrasonic waves reflected by the soil until their detection;
ii) emitting electromagnetic radiation with at least one radiation source in the direction of the soil into a surface region; in which mechanical weed removal is carried out or is to be carried out, and registering the intensity of electromagnetic radiation reflected or scattered there with at least one optical detector arranged on the hoeing machine, and determining with the registered intensity whether a sufficient tillage has taken place, which has resulted in weed removal, and/or whether too much dust has been whirled up;
iii) detecting and taking into account during monitoring of shear and/or bending stresses at individual tools with strain gauges and comparing them with calibration values that have been determined at the corresponding tool in case of a correct removal of weeds; and
iv) detecting airborne sound waves emitted by at least one tool as a result of weed removal with at least one microphone and comparing the detected airborne sound spectra with an electronic evaluation unit with airborne sound spectra registered in advance during faultless weed removal.

2. The method according to claim 1, characterized in that at the individual tools, vibrations are detected by detectors with respect to their amplitude and frequency resolution, and/or speeds of rotating elements at tools are registered and taken into account for a functionality monitoring.

3. The method according to claim 2, characterized in that similar measured values registered at all tools at same points in time are compared with one another.

4. The method according to claim 1, characterized in that a calibration run is carried out before the start of monitoring and/or at least one calibration run is carried out during processing.

5. The method according to claim 1, characterized in that the intensity of electromagnetic radiation emitted by the at least one radiation source and scattered and reflected by the soil is registered by a plurality of optical detectors arranged in the region of a tool on the hoeing machine and, if a predetermined threshold value is exceeded, it is determined by optical detectors with a predeterminable number that sufficient processing has been carried out with the respective tool.

6. The method according to claim 1, characterized in that determining the intensities is carried out with a plurality of optical detectors arranged at a distance from each other and arranged in a row and/or column arrangement or along circular rings with different diameters.

7. The method according to claim 1, characterized in that images of regions on the tools are monitored with at least one camera and the images are transmitted to a monitoring center in the event of a malfunction registered with a detector, so that measures can be initiated to remedy the malfunction.

8. The method according to claim 2, characterized in that a device is present on tools in each case by means of which, when a malfunction is registered with the tool brought into a rest position, vibrations are coupled into the respective tool or its surroundings on the hoeing machine with an amplitude at which a release of a clogging or a jamming of weeds or objects is achieved by the respective tool.

9. The method according to claim 8, characterized in that a rotating mass with an unbalance is used as the device for coupling vibrations.

10. The method according to claim 1, characterized in that, in the event of clogging, the hoeing machine is raised and, after a short run, lowered again.

11. The method according to claim 1, further comprising transmitting registered signals of at least one camera present on the hoeing machine to an electronic image evaluation unit, and using data evaluated with the electronic image evaluation unit to monitor whether weed plants have been sufficiently covered with soil after being burrowed, have been torn out of the soil and/or whether cultivated plants have been damaged.

12. The method according to claim 1, further comprising using an electronic control system to adjust the travel speed, the working depth and/or the distances between the tools when at least one predetermined threshold value of a measurement signal registered is exceeded or not reached.

13. The method according to claim 1, characterized in that the force and/or power required for advancement of the hoeing machine is/are determined and thus the working depth of the tools is/are affected and/or the travel speed is/are adapted to soil conditions in a locally defined manner
